(19) United States
(12) Patent Application Publication
(10) Pub. No.: US 2012/0259291 A1
(43) Pub. Date: Oct. 11, 2012

(54) MEDICAL FLOW DEVICE FOR MULTI-LUMEN CATHETERS

(75) Inventors: Raymond Lareau, Westford, MA (US); Stephen Engelhard, Queensbury, NY (US); Eric Houde, Glens Falls, NY (US)

(73) Assignee: Navilyst Medical, Inc., Marlboro, MA (US)

(21) Appl. No.: 13/080,926

(22) Filed: Apr. 6, 2011

(51) Int. Cl. A61M 25/14; A61M 25/16; A61M 25/00

(52) U.S. Cl. 604/247; 604/523; 604/533

(57) ABSTRACT

An apparatus for providing access to a target site in a living body, comprises a first elongated member including a proximal opening to a first lumen and a branching point at a distal end thereof and a second lumen open to the first lumen at the branching point, the second lumen extending to a second lumen distal opening at a first connector, the first connector configured to engage a proximal end of a first lumen of a multi-lumen catheter in combination with a third lumen open to the first lumen at the branching point, the third lumen extending to a third lumen distal opening at a second connector, the second connector configured to engage a proximal end of a second lumen of a multi-lumen catheter.

### BACKGROUND

Catheters are generally used for the introduction of fluids to and/or the withdrawal of fluids from living bodies. Venous Access Devices (“VAD”s) such as peripherally inserted central catheters (“PICC”s) are formed as elongated flexible tubes used to deliver fluids, nutrients, etc. over an extended period of time or to aspirate blood from the patient for diagnosis. Presently available VADs are formed with single or multiple lumens to deliver, for example, different agents to different target sites in a living body or to provide simultaneous infusion and withdrawal, as those skilled in the art will understand. Each of these lumens may be connected to a separate infusion or withdrawal source to affect flow therethrough.

### SUMMARY OF THE INVENTION

In one aspect, the present invention is directed to apparatuses for providing access to a living body, comprising a first elongated member including a proximal opening to a first lumen and a branching point at a distal end thereof and a second lumen open to the first lumen at the branching point, the second lumen extending to a second lumen distal opening at a first connector, the first connector configured to engage a proximal end of a first lumen of a multilumen catheter in combination with a third lumen open to the first lumen at the branching point, the third lumen extending to a third lumen distal opening at a second connector, the second connector configured to engage a proximal end of a second lumen of a multi-lumen catheter. In another aspect, the present invention is directed to a flow control element within a multi-lumen catheter, which flow control element provides separate fluid flow paths for the multiple lumens of a catheter during low pressure or low-flow conditions, but which selectively places the multiple lumens of the catheter in fluid communication with one-another during high pressure or high-flow conditions.

### BRIEF DESCRIPTION OF THE DRAWINGS

FIG. 1 shows a perspective view of a first exemplary device according to an embodiment of the invention.

FIG. 2 is a schematic view of flow balancing element of a device in accordance with one aspect of the invention.

### DETAILED DESCRIPTION

The present invention may be further understood with reference to the following description and the related appended drawings. The present invention describes a device for use with a multi-lumen catheter for infusion of agents into a target structure in a living body (e.g., a blood vessel, body cavity, etc.) and/or the removal of fluids or other materials therefrom. The flow device of the present invention facilitates the flow of high pressure fluids from a single fluid source into both lumens of a dual-lumen catheter, making possible the use of higher flow rates than available through either lumen of the catheter. Although exemplary embodiments of the present invention are described with respect to dual-lumen catheters and dual-lumen flow devices, the system 100 may be formed with any additional number of lumens without deviating from the spirit and scope of the present invention.

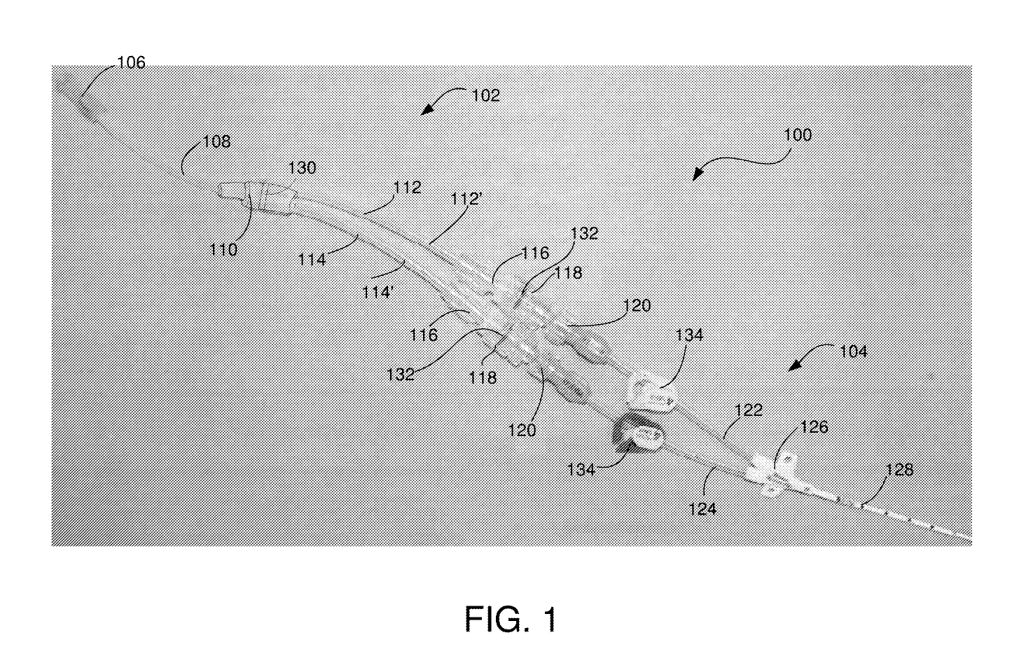

FIG. 1 depicts the system 100 according to the present invention comprising a flow device 102 connected to a dual-lumen catheter 104, a distal end (not shown) of which is open to a target anatomical structure when implanted in an operative configuration. A port 106 at a proximal end of the flow device 102 is configured in a conventional manner to engage a single injection source (not shown). Alternatively, the port 106 is attached to a device for withdrawing fluid from the body. The port 106 is connected to a single lumen 108 tube which extends distally to an adaptor 110 which separates flow from the lumen 108 into the lumens 112′, 114′ of two separate tubes 112, 114, respectively. The adaptor 110 may be of any suitable shape to achieve the desired flow separation including, without limitation, Y-shaped, H-shaped, or in the form of a manifold. In alternate embodiments, the adaptor 110 may further comprise a stopcock 130 or a pressure activated valve (not shown) to control the flow of fluid through one or more of the lumens 112′, 114′. The adaptor 110 provides a connection between the single lumen 108 and each of lumens 112′, 114′. The lumens 112′, 114′ are preferably formed with the same dimensions but, alternatively, can be formed with different dimensions and/or features to affect flow therethrough.

A distal end of each of the tubes 112, 114 includes a luer port 116 configured to engage a respective luer port of a dual-lumen catheter 104. Specifically, the luer ports 116 may be formed, for example, as male luer ports with collars 118 that can be rotated to lock against female luer ports 120 of tubes 122, 124 of the dual-lumen catheter 104, as those skilled in the art will understand. Furthermore, each of the tubes 112, 114 is preferably formed of a flexible material and of a length permitting a degree of movement sufficient to facilitate connection of the flow device 102 and the dual-lumen catheter 104. As would be understood by those skilled in the art, the dual-lumen catheter 104 may include pinch valves 134 or other means for sealing the lumens when the catheter 104 is not in use. Distal ends of each of the tubes 122, 124 are fluidly connected to a connector 126 which draws each of the tubes 122, 124 into a single, dual lumen, outer tube 128 insertable into the body in the operative configuration, as is known to those of skill in the art.

In an alternate embodiment of the present invention, the system 100 may comprise a fluid management manifold (not shown) formed with a predetermined number of male luer ports that are attachable to female luer ports of a multi-lumen catheter. The fluid management manifold may facilitate the attachment of the multi-lumen flow device 102 of the present invention to a multi-lumen catheter irrespective of the number of lumens each element comprises.

In still another embodiment, each of the lumens 112′, 114′ may further comprise a valve 132 located, for example, in the luer port 116 and configured to prevent any back-flow of fluid from the catheter 104, as those skilled in the art will understand. For example, one or more of the valves 132 may be constructed to remain closed to prevent flow therethrough at all times during which a predetermined parameter is not met or exceeded (e.g., flow rate, velocity, pressure, etc.). For example, the valves 132 may be pressure activated so that, when a fluid pressure within the one of the lumens 112′, 114′ corresponding to the particular valve 132 is less than a predetermined magnitude, the valve 132 remains closed sealing the corresponding lumen 112′, 114′. When the fluid pressure within this one of the lumens 112′, 114′ exceeds the predetermined magnitude, the valve 132 opens to permit fluid flow therethrough. Those skilled in the art will understand that, depending on the characteristics of the individual lumens of the catheter 104 and any valves therein, different threshold pressures may be set for the valves 132 in the corresponding lumens 112′, 114′ to which these lumens will be connected. For example, where the pressure in the lumen 108 and the setting of any flow balancing/biasing features is such that the pressure in the lumen 112′ is greater than the threshold for the corresponding valve 132 while the pressure in the lumen 114′ is lower than the threshold for its valve 132, fluid will flow only through the lumen 112′ and the corresponding lumen of the catheter 104 while no fluid flows through the lumen 114′ and the corresponding lumen of the catheter 104. If the pressure within the lumen 108 rises to the point at which the pressure in the lumen 114′ exceeds its threshold level, its valve 132 opens to permit flow therepast. Thus, the valves 132 may be configured to permit the use of one lumen of a multi-lumen catheter when flow through the flow device 102 meets a first predetermined condition and permits the use of more than one lumen when the flow meets a second predetermined condition. The valves 132 thus act as a safety precaution to prevent overpressurization within the catheter 104, as those skilled in the art will understand.

In another embodiment of the present invention, one or more of the pressure-activated valves 132 may be replaced by a transducer/valve combination (not shown) that function in a manner substantially similar to the valves 132 described above. Specifically, the transducers may be configured to monitor one or more parameters within the lumens 112′, 114′ as described above and provide a signal to a control system (not shown) that opens a valve in the corresponding lumen when the parameter in that lumen meets the predetermined conditions. It is noted that the aforementioned embodiments are exemplary only and that the valves 132 of the device 100 may be configured to permit flow at any predetermined rates and parameters without deviating from the scope of the present invention.

In another embodiment, at least one of the lumens 112′, 114′ may be provided with an overpressure safety mechanism such as a rupture disk (not shown) that prevents overpressurization of the catheter 104, as those skilled in the art will understand. Specifically, if a pressure within one or both of the lumens 112′, 114′ exceeds a predetermined threshold, the rupture disk bursts externally of the body, preventing damage to the catheter 104 and any leakage within the body of the fluids being transferred.

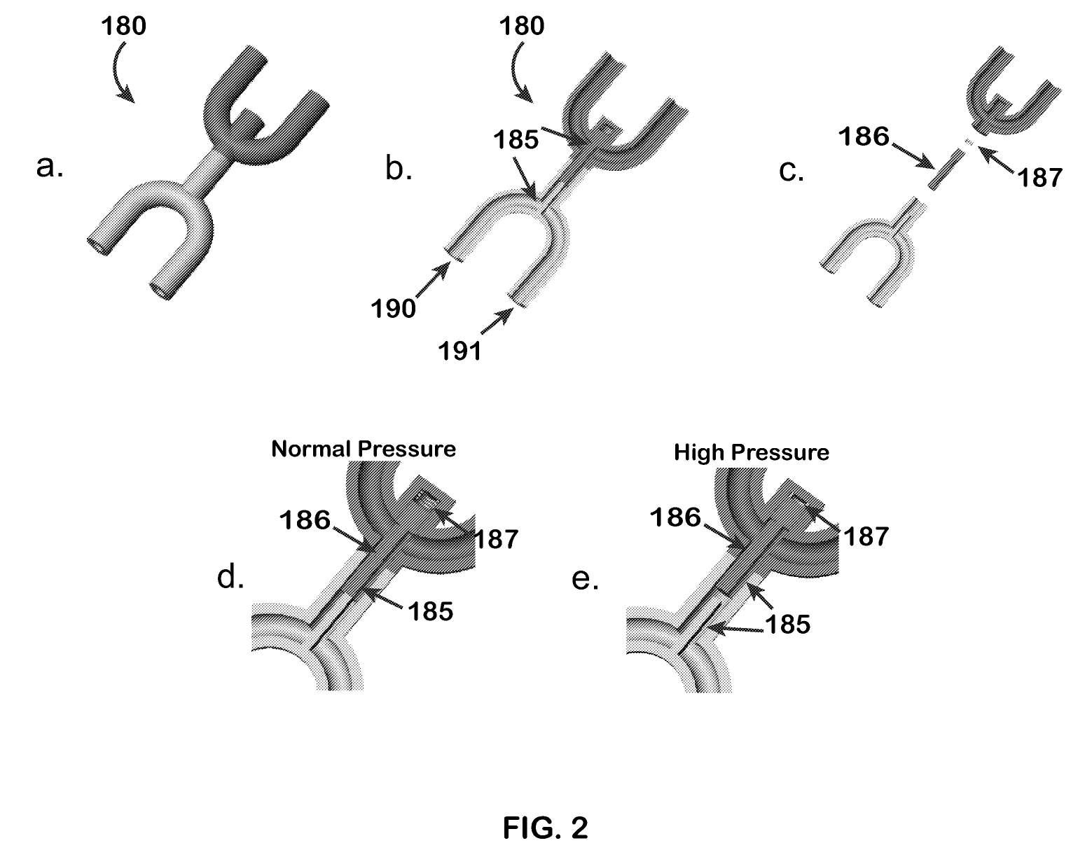

In another aspect of the present invention, a flow-balancing feature is provided within a multi-lumen catheter to facilitate power injection. In the exemplary embodiment depicted in FIG. 2, the flow balancing feature comprises an H-shaped connector 180 with two lumens, 190, 191, which lumens are in fluid communication with two lumens of a catheter. The lumens 190, 191 of the H-shaped adaptor 180 share a common wall 185, which wall includes a flow control element 186. In the embodiment shown in FIG. 2a, the flow control element includes a spring 187 and a surface which is acted upon by pressures in lumens 190, 191. As shown in FIG. 2d, when the fluid pressures in lumens 190 and 191 are below a threshold level, the flow control element is not activated, and the common wall remains in place between the lumens 190, 191. However, when the pressures in either of lumens 190 or 191 exceeds a pre-determined threshold level, as shown in FIG. 2e, the flow control element 186 is activated, the wall 185 between lumens 190, 191 is breached, and the lumens 190, 191 are placed into fluid communication with one another.

In another aspect, the present invention includes a method of using a flow-control element to perform power injections using a catheter. In certain embodiments, users can optionally utilize a single luer input of a multi-lumen catheter to perform power injections. As discussed above, and as shown in FIG. 2e, when the pressure in the fluid flow path being utilized for power injection exceeds a threshold pressure, the flow control element 186 is activated, and the fluid being power injected will flow into the second fluid flow path. To prevent reflux towards the proximal luer of the catheter, a check-valve or clamp can be utilized.

It is noted that the embodiments shown are shown for illustrative and descriptive purposes only and are not intended to describe the bounds of the present invention which is to be limited only by the scope of the claims appended hereto. There are many modifications of the present invention which will be apparent to those skilled in the art without departing from the teaching of the present invention. Various modifications may be made to the size and shape of the flow device without deviating from the spirit and scope of the present invention.

What is claimed is:

1. An apparatus for providing access to a target site in a living body, comprising:
a first elongated member including a proximal opening to a first lumen and a branching point at a distal end thereof;
a second lumen open to the first lumen at the branching point, the second lumen extending to a second lumen distal opening at a first connector, the first connector configured to engage a proximal end of a first lumen of a multi-lumen catheter;
a third lumen open to the first lumen at the branching point, the third lumen extending to a third lumen distal opening at a second connector, the second connector configured to engage a proximal end of a second lumen of a multi-lumen catheter.

2. The apparatus of claim 1, wherein distal ends of each of the lumens comprise male luer ports.

3. The apparatus of claim 1, further comprising a flow biasing element to direct a first desired portion of fluid flow through the first lumen into the second lumen and a second desired portion of fluid flow through the first lumen into the third lumen.

4. The apparatus of claim 2, wherein the flow biasing element may be adjusted by a user to alter the first and second desired portions.

5. The apparatus of claim 4, further comprising:
a Y-shaped connector at the branching point; and
wherein the second and third lumens extend distally from the Y-shaped connector through second and third elongated members, respectively.

6. The apparatus of claim 1, further comprising a first flow control valve in the second lumen, the first flow control valve permitting fluid flow through the second lumen only when a parameter in the second lumen is at least a first threshold level.

7. The apparatus of claim 6, wherein the parameter is a fluid pressure within the second lumen and wherein, the first flow control valve permits fluid flow through the second lumen only when a pressure in the second lumen is at least the first threshold level.

8. The apparatus of claim 8, further comprising a second flow control valve in the third lumen, the second flow control valve permitting fluid flow through the third lumen only when a pressure in the third lumen is at least a second threshold level.

9. The apparatus of claim 8, wherein the first and second threshold levels are substantially equal to one another.

10. The apparatus of claim 8, wherein the first and second threshold levels are different from one another.

11. The apparatus of claim 7, wherein the first flow control valve is controlled by a controller based on data regarding fluid pressure within the second lumen received from a first transducer.

12. The apparatus of claim 4, wherein the flow biasing element includes a stopcock.

13. The apparatus of claim 1, wherein the mechanism is one of a transducer coupled to a control system and a pressure-activated valve.

14. The apparatus of claim 6, wherein the predetermined parameter is of flow rate, velocity and pressure.

15. The apparatus of claim 1, further comprising an overpressure element configured to open when a predetermined maximum pressure is exceeded to release fluid from the apparatus via an opening other than the second and third lumen distal openings.

16. An apparatus, comprising:
a first and second set of lumens for fluid flow, each set comprising at least one of said lumens; and
at least one valve sensitive to a predetermined fluid flow parameter; wherein the valve channels fluid flow into the second set of lumens during flow conditions above the predetermined fluid flow parameter.

17. The apparatus of claim 16, wherein there is no fluid flow through the second set of lumens during flow conditions below the predetermined fluid flow parameter.

18. The apparatus of claim 17, wherein there is fluid flow in both the first and second sets of lumens during flow conditions above the predetermined fluid flow parameter.

19. The apparatus of claim 16, wherein the fluid flow parameter is a pressure.

20. The apparatus of claim 16 wherein the fluid flow parameter is a flow rate.

21. The apparatus of claim 16 wherein the valve includes a pressure-sensing element.

22. The apparatus of claim 16 wherein the valve includes a flow-rate sensing element.

23. The apparatus of claim 16 wherein the valve includes a flexible membrane having one or more slits therethrough.
